(19) United States
(12) Patent Application Publication
(10) Pub. No.: US 2009/0221528 A1
(43) Pub. Date: Sep. 3, 2009

(54) Therapeutic micro nutrient composition for lipolysis and sclerosis

(76) Inventors: Teresa Ann Denney, Honoluu, HI (US); James Robert Deluze, Honolulu, HI (US)

Correspondence Address: James Robert Deluze, 611 University Ave. Apt. 106, Honolulu, HI, 96826, US

(21) Appl. No.: 12/316,959

(22) Filed: Dec. 16, 2008

Related U.S. Application Data
(63) Continuation-in-part of application No. 10/881,170, filed on Jun. 28, 2004, now abandoned.

(51) Int. Cl. A61K 31/66

(52) U.S. Cl. 514/75

(57) ABSTRACT

An improved formulation and method for the removal of subcutaneous fat deposits in a human in need of such treatment. It also induces an inflammatory reaction which treats disorders of tissue adhesion which often accompany subcutaneous fat deposits or are created in the process of removal of subcutaneous fat deposits. This inflammatory reaction also limits the extent of the effect of the injected medication to a localized area. A lecithin and aqueous sodium chloride based biphasic injection dosage formulation is disclosed which is applicable to subcutaneous, intramuscular, and intravenous administration. Additionally, a program based approach to the treatment of subcutaneous fat deposits which includes injections of this formulation, application of compression garments, diet modification, and exercise is described. The formulation is characterized in that it comprises an adjustable acidifying agent to set pH, an antioxidant, a sclerosing agent, and a stabilizer. It is further characterized in that it includes liposomes, and that the components of these liposomes are therapeutic in the treatment of several human ailments. It is also efficacious in the treatment of striae albicantes, striae atrophicae, cellulite, and decreased skin turgor.

### CROSS-REFERENCE TO RELATED APPLICATIONS

“This application claims the benefit of Regular patent application Ser. No. 10/881,170, filed 2004 Jun. 28 by the present inventors.

### BACKGROUND

1. Field of Invention

The present invention relates to a biphasic aqueous and lipolytic combination of phosphatidylcholine, a sclerosing compound, a bile acid or a bile salt, and water. In particular, the invention relates to a biphasic injectable dosage form of phosphatidylcholine and other micro nutrients for the lipolysis of subcutaneous fat. It further relates to the to a biphasic injection dosage form of a sclerosing compound for the treatment of disorders of tissue adhesion. These disorders of tissue adhesion accompany subcutaneous accumulations of fat or are caused by the procedures intended for removing such subcutaneous fat accumulations. This combination of actions results in medicaments for removing subcutaneous accumulations of fat and lead to regression of diet-resistant fat pads. At the same time it restores proper tissue adhesion, thus removing or preventing these disorders of tissue adhesion. This prevents post procedure tissue looseness and flaccidity resulting in undesirable wrinkling and hanging of skin.

2. Description of Prior Art

Aqueous sodium chloride is commonly used as an injectable, parenteral, or enteral dosage formulation for medical treatment and as a delivery agent for other therapeutic agents.

Phosphatidylcholine is also used as an injectable, parenteral or enteral dosage formulation for treatment of various disorders.

Bile salts and bile acids are also used in an injectable, parenteral or enteral dosage formulation for treatment of various disorders.

Thereafter, inventors created an oral dosage formulation of micro nutrients for treatment of severe trauma, burns, and critical illness. U.S. Pat. No. 6,391,332 patent to Somerville and Sherratt (2002), incorporated herein by reference, and discloses a micro nutrient formulation containing water soluble and lipid soluble micro nutrients for oral and feeding tube administration.

Thereafter, inventors created a subcutaneous dosage formulation of erythropoietin utilizing phosphatidylcholine and other micro nutrients as an erythropoietin carrier. U.S. Pat. No. 6,645,522 to Naeff, Delmenico, Wetter, and Floether (2003), incorporated herein by reference, discloses a liposome based formulation of erythropoietin comprising: (a) erythropoietin; (b) a lipidic phase comprising: (i) lecithin; (ii) a charged lipid; and (iii) cholesterol; and (c) a phosphate buffer.

Thereafter, inventors created a subcutaneous dosage formulation of a bile acid, a bile salt, or a phospholipid and other micro nutrients as an medicament for removing subcutaneous accumulations of fat. U.S. patent 20050143347 to Boderke; et al. (2005), incorporated herein by reference, discloses a aqueous preparation comprising a) at least one phospholipid or b) at least one bile acid, c) a component assisting degradation of fat and d) water.

### BACKGROUND OF THE INVENTION

Lecithin is a phospholipid which serves as a principal factor involved in the transport, regulation, and metabolism of fatty substances. It is a fatty food substance. It is a structural component of every cell in the body. It is an important component of cell membranes. It has been used as an effective treatment agent in the treatment of hypercholesteremia, hypertriglyceridemia, alcoholic hepatic steatosis, and xanthelasma. The phospholipid is administered orally or parenteral as either an intravenous (IV) or subcutaneous (SC) injection.

Presently, lecithin is injected SC for the reduction of subcutaneous fat deposits. This procedure was discovered by Brazilian dermatologist Patricia Rittes and is most commonly called Lipo-Disolve. In April 1999 the FDA approved a Baxter Healthcare Corporation product, Cernevit-12, which contains lecithin for injection. This product is a vitamin delivery system for parenteral nutrition.

Hexsel et al. Journal of Drugs in Dermatology (2003, 2:5 pages 511-518), incorporated herein by reference, on page 511 listed the following as exclusion criteria against using this procedure: moderate to severe loose skin or flaccidity. On page 517 a limitation of 10 ml per treatment session with a rest period between treatments of at least 10 days between applications was cited. The same page listed several factors which may determine either a lack of success or of greater risk of side effects. These included: total doses above those considered safe, excessive injection volume at each point, and superficial injections less than 1 cm below the epidermal surface.

Boderke, et al. in U.S. patent 20050143347 recommends from 5 mg to 500 mg dosage per injection. This is consistent with the dosage recommendations of Hexsel et al.

“At present, subcutaneous accumulations of fat or proliferation's of adipose cells such as lipomas or lipedemas are treated by surgical means through liposuction or direct surgical removal. Treatment measures of these types of are associated with the known complications or risks caused by anesthesia, local reactions and possible infections, and in some circumstances require admission to a hospital ward.”

“Aqueous preparations comprising at least one phospholipid and/or at least one bile acid are known for various applications. Thus, these systems are employed for example in the cosmetics sector or for manufacturing pharmaceutical products. These systems are in some cases notable for forming spherical vesicles, which are also referred to as liposomes. Said liposomes have a double lipid membrane boundary to the outside and contain an aqueous phase in their interior. Aqueous preparations comprising at least one phospholipid, at least one bile acid and water are described for example in the European patent application EP 0 615 746, incorporated herein by reference. A commercially available product is Essentiale.RTM. N i.V. (Rote Liste, March 2003), which is an aqueous preparation comprising phospholipids, bile acid, riboflavin, alpha-tocopherol, ethanol and water and is approved for the treatment of, for example, hepatopathies, acute and chronic hepatitis, fatty degeneration of the liver or hepatic necrosis.”

“It is known that fatty degeneration of the liver involves an excess fat content of the liver parenchyma (deposition of fat in droplet form) which may lead to cell necroses, inflammation or fibrosis. Fatty degeneration of the liver occurs if the production or intake of fat exceeds the degradation thereof. Fatty degeneration of the liver is present if more than half of liver cells have fatty deposits. It is associated for example with obesity, protein deficiency, diabetes mellitus, chronic alcoholism or as a consequence of necroses after hepatotoxins. Intravenous administration of the medicament Essentiale.RTM. can have a beneficial effect on the progress of these liver disorders.”

“It is reported that fat pads like those occurring under the eyes, on the abdomen or on the hips of overweight people shrink, and there are said to be esthetic improvements in the appearance of the treated people, if these people received subcutaneous injection of Lipostabil.RTM. N i.V. (Patricia Guedes Rittes, The Use of Phosphatidylcholine for Correction of Lower Lid Bulging Due to Prominent Fat Pads, Dermatol. Surg. 2001; 27: 391-392, incorporated herein by reference). Lipostabil.RTM. N i.V. is a solution for injection which comprises soybean phospholipids, deoxycholic acid, sodium chloride, sodium hydroxide, DL-alpha-tocopherol, ethanol, benzyl alcohol, ethanol and water.”

“In the attempt to find effective compounds for non surgical removal of subcutaneous accumulations of fat, it has now surprisingly been found that subcutaneous administration of the aqueous preparations, employed according to the invention, of this pharmaceutical form Essentiale.RTM. N i.V., which have to date been used only for the treatment of liver disorders, also leads to regression of depot fat in the body. Lipolysis of the adipose tissue occurs, and the zone of adipose tissue regresses.”

While these formulations have been proven to be successful, further improvement for the treatment of subcutaneous fat deposits has been desired. The reduction of subcutaneous fat can leave the skin loose. The efficacy of the procedure could be significantly improved. Hence, there is a need for an improved phospholipid formulation for the treatment of subcutaneous fat deposits.

Boderke states: “Daily doses required for the treatment of an adult patient are, depending on the size of the treated adipose tissue, on administration of solutions for injection from 5 mg to 500 mg, preferably 250 mg to 500 mg, per injection, based on the phospholipid.” Standard lecithin ampules for oral consumption contain up to 1000 mg in an ampule of about 2-3 ml of fluid. Based on the dilutions described by Boderke the total dosage for a given treatment session would be about 20 ml. With our invention, it is not uncommon to give a single session total dosage of over 100 ml during a single session. Additionally, the self limiting action of the sclerosis induced inflammation allows for the time between treatments sessions to be reduced to one to two day intervals without complications.

Accordingly, attempts have been made to provide an improved formulation of phospholipid which increases skin tone and proves to be more efficacious. At the same time the formulation should be stable and of the correct hydrogen ion concentration (pH).

A biphasic formulation comprises an aqueous phase and a lipidic phase. This provides for the solubility of both lipid and aqueous soluble components. This formulation then can be a vehicle for the transport of both lipid and water soluble substances to the targeted treatment area.

The properties of such a biphasic formulation will provide conditions which favor the formation of liposome. Liposome are small vesicles comprising amphipathic lipids arranged in spherical bilayers. Liposome may contain many concentric lipid bilayers separated by aqueous channels (multilamellar vesicles or MLVs), or alternatively, they may contain a single membrane bilayer (unilamellar vesicles), which may be small unilamellar vesicles (SUVs) or large unilamellar vesicles (LUVs). The lipid bilayer is composed of two lipid monolayers having a hydrophobic “tail” region and a hydrophilic “head” region. In the membrane bilayer, the hydrophobic “tails” of the lipid monolayers orient towards the center of the bilayer, whereas the hydrophilic “heads” orient towards the aqueous phase.

Liposomes may be used to encapsulate a variety of materials by trapping hydrophilic compounds in the aqueous interior or between bilayers, or by trapping hydrophobic compounds within the bilayer. In addition, liposomes may be used to deliver biologically active materials which are at the same time components of the liposome itself. Such is the result of the formation of liposomes from phospholipids including lecithin and it's pharmaceutically acceptable derivatives.

The goal of this present invention therefore was to provide a parenteral formulation suitable for the treatment of subcutaneous fat deposits, provides for pH adjustment, has stability, increases skin turgor, permits higher dosage levels, reduces side effects, allows decreased time before successive administrations, and is not contraindicated in areas of moderate to severe loose skin or flaccidity.

### SUMMARY

A biphasic phospholipid based composition comprising:

a) an effective amount of an active ingredient comprising a phospholipid selected from the group consisting of: 3-sn-phosphatidylcholine, soya (Phospholipon 90), 3-sn-phosphatidylcholine, hydrogenated soya (Phospholipon 90H), 3-(3sn)-phosphatidyl)glycerol soya (Phospholipon G), (Phospholipon 90G), dimyristoylphosphatidylglycerol, lysophosphatidylcholine or dipalmitoylphosphatidylglycerol, and physiologically tolerated salts thereof, or a mixture of these compounds. This active ingredient or active ingredients having the biological properties of causing the destruction of adipose cells and the lipolysis of the fatty material contained within the treated adipose tissue; b) a bile acid or a bile salt wherein the bile acid employed is selected from the group consisting of deoxycholic acid, cholic acid, lithocholic acid, chenodeoxycholic acid, hyodeoxycholic acid, trihydroxycoprostanic acid, ursodeoxycholic acid, taurocholic acid and glycocholic acid, and the physiologically tolerated salts thereof, or a mixture thereof. That this bile acid or bile salt having the properties of being an emulsifier; c) a sclerosing component; d) water; e) a clarifying component; f) an alkalizing agent; g) and an acidification agent.

A biphasic phospholipid based composition comprising:

a) a lipidic phase comprising:
(i) lecithin or hydrogenated lecithin;
(ii) phosphatidylcholine or hydrogenated phosphatidylcholine;
(iii) optional lipid soluble components as described below in (c); and
   b) an aqueous phase comprising:
(i) water;
(ii) sodium chloride;
(iii) benzyl alcohol;
(iv) hydrochloric acid;
(v) a bile acid or a bile salt;
(vi) sodium hydroxide
(vii) optional aqueous soluble components described below in (d); and
   c) lipid soluble biologically active substances as added to individualized treatment regimens as further selected from the group consisting of nutrients, micro nutrients, vitamins, and drugs; and
   d) aqueous soluble biologically active substances as added to individualized treatment regimens as further selected from the group consisting of nutrients, micro nutrients, vitamins, and drugs.

In accordance with the invention, the selected components of the lipid phase are mixed with the selected components of the aqueous phase. The relative amounts of the compounds mixed is predetermined. The mixing of these lipid and water soluble components favors the formation of liposomes.

Liposomes may be used to encapsulate a variety of materials by trapping hydrophilic compounds in the aqueous interior or between bilayers, or by trapping hydrophobic compounds within the bilayer. As such, they are particularly useful to deliver biologically active materials by encapsulating compounds which exhibit poor aqueous solubility or which exhibit unacceptable toxicity at therapeutic dosages.

In addition, liposomes may be used to deliver biologically active materials which are at the same time components of the liposome itself. Such is the result of the formation of liposomes from phospholipids including lecithin and it's pharmaceutically acceptable derivatives.

In addition, a method of treatment for subcutaneous fat deposits has been developed which includes the following components:

(a) injection of the biphasic phospholipid based parental composition;
(b) application of a compression garment;
(c) a predetermined exercise program;
(d) a predetermined diet regimen.

In accordance with the present invention, it has been discovered that this biphasic phospholipid based parental composition described herein exhibits improved efficacy in the treatment of subcutaneous fat deposits. As further advantages, the presence of the sodium chloride acts as a mild sclerosing agent. This sclerosing action has the physiological function of increasing the adherence of subcutaneous layers of skin and fascia resulting in increased skin turgor. It also stimulates fibroblasts to reformat the facial components of the sub cutaneous tissue structures. It has also been discovered that, quite unexpectedly, that this combination of fat reduction and mild sclerosing action produces a medicament that is efficacious for the treatment of cellulite, subcutaneous derangements of fat distribution, adipose tissue disorders, Dercum's disease, Madelung's neck, lipedema, piezogenic nodules, xanthelasma, and striae albicans.

The term “subcutaneous derangements of fat distribution” means adipose tissues in the body of humans and mammals which occur as genetically related or food-related depot fat in the form of localized fat pads and can be regarded as esthetically disturbing critical zones such as abdomen, buttocks, hips, knee, calves, thighs, upper arm, chin, cheeks. They may also involve dystopic proliferation (benign proliferations of the fat cells such as lipomas).

The term “adipose tissue disorders” means for example the following disorders: lipomas are adipose tissue tumors, which are benign, slow-growing, usually spherical, possibly pedunculated (lipoma pendulum) or even villous (lipoma arborescens, for example of the synovial villi) mesenchymal tumors composed of enlarged adipose tissue cells, preferentially in a subcutaneous cell tissue, possibly with central ossification (lipoma ossificans), becoming mucoid (lipoma myxomatodes) or calcifying (lipoma petrificans), also with increased connective tissue and capsule formation (lipoma fibrosum), neoangiogenesis (lipoma teleangiectodes), rarely showing malignant degeneration (lipoma sarcomatodes, liposarcoma). They are to be categorized as pathological because they grow and their connective tissue envelope may be painful per se, as well as the compression derived therefrom on blood vessels, which may cause neuralgia.

Dercum's disease, called lipomatosis dolorosa, is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels and with which additional compressive and irritant effects are exerted on the peripheral sensory nerves, so that these patients display an extremely painful sensitivity to touch. Over the course of several years up to decades there is formation of irregular fatty nodules in disseminated locations underneath the dermis, which becomes thinner during the aging process, some of which nodules have painful and highly dysesthetic characteristics.

Madelung's neck (Lanois-Bensaude syndrome) is an adipose tissue inflammation with adipose tissue proliferation in which a dystrophic adipose tissue tumor formation is accompanied by subcutaneous scarlike connective tissue compaction. In such cases, surgical procedures can often be only partially successful, because essential anatomic structures are involved in this process and the disorder is manifested essentially in the region of the head, neck and shoulders.

Lipedema is a painful adipose tissue swelling which occurs especially on the lower legs of women and shows a progressive course and characteristics with increasing age.

Piezogenic nodules are nodules on the edges of the hands and the heels which are caused by pressure and occur as multiple adipose tissue hernias, mainly in the medial region of the heel in obese people. They are usually defects in the septation of the subcutaneous adipose tissue which are regarded by patients as cosmetically or functionally disturbing.

Xanthelasma is a pale yellow, slightly raised plaque-like deposit of cholesterol in the region of the eyelids. They are soft and easily displaceable and usually occur symmetrically on both eyes. It is caused by local derangements of lipid metabolism. Post menopausal women are affected particularly frequently. Diabetes mellitus and elevated blood lipid levels are also associated with an increased risk of developing it. Xanthelasmas may cause psychological stress because of their appearance.

The above mentioned adipose tissue disorders show, in contrast to the food-related lipohypertrophy (which is also followed by a deposition of fat in the sense of the derangement of fat distribution), tissue conditions or entities which can be pathologically differentiated unambiguously and which can be described by histological parameters of scarring and inflammation, but also by connective tissue encapsulations and by changes in the histological adipose tissue morphology itself.

Cellulite is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels. Over the course of several years up to decades there is formation of irregular fatty nodules in disseminated locations underneath the dermis, which becomes thinner during the aging process, some of which nodules have painful and highly dysesthetic characteristics.

The term striae atrophicae means linear, depressed, atropic, pinkish or purplish, scarlike lesions that later become white, on the abdomen, breasts, buttox, and thighs. They are due to weakening of the elastic tissues. Dorland's Illustrated Medical Dictionary, 26th ed. incorporated herein by reference.

### OBJECTS AND ADVANTAGES

Accordingly, besides the objects and advantages of this biphasic micro nutrient dosage formulation described in our above patent, several objects and advantages of the present invention are:
(a) to circumvent the bodies metabolic adjustments as part of the feast or famine cycle.
(b) to reduce the numbers of adipose cells in treated areas.
(c) to reduce the amount of adipose issue in treated areas.
(d) to provide an injectable, parenteral or enteral nutrient delivery means for aqueous soluble substances.
(e) to provide an injectable, parenteral or enteral nutrient delivery means for lipid soluble substances.
(f) to provide a sclerosing action in affected tissue.
(g) to provide treatment for disorders of tissue adhesion.
(h) to localize the biological effects of the preparation to tissue areas in the local proximity of the injection site.
(i) to limit the toxic effects of lysolecithin.
(j) to smooth and soften the skin in areas of previous liposuction.
(k) to smooth and soften the skin in areas of cellulite.

### DESCRIPTION OF INVENTION

A method for reduction of sub adipose tissue in humans comprising injection of an effective amount of a biphasic dosage formulation based on lecithin and aqueous sodium chloride. It is in amount effective to stimulate lipase production in adipose tissue. It is in amount effective to produce an inflammatory reaction within the treated tissue. Lipase, and it's related compounds, stimulate the lysis, destruction, and reduction of the amount of adipose cells. Lipase also stimulates the lipolysis of fatty material contained within adipose tissue. The inflammatory reaction is a treatment of disorders of tissue adhesion.

This invention encompasses a systematic means of reduction of adipose tissue. It incorporates the biphasic dosage formulation based on lecithin and aqueous sodium chloride, the use of a compression garment, exercise management and counseling, and diet management and counseling. This multifaceted approach increases the successful removal of unwanted adipose tissue. This biphasic injection formulation is called Lipolyte.

Obesity is a serious medical problem resulting is significant morbidity and mortality. Many people spend hours exercising and try all kinds of diet regimens, but the obesity remains.

The underlying problem is that the body has an internal set point of how much fat composition is optimal. This set point is basically determined by the numbers of adipose cells present. This was determined by both genetic and environmental factors. The environmental factors include the behavior of a persons mother during pregnancy, behavior patterns during growth spurts during early childhood and at puberty, and continuing behavioral patterns into adulthood.

When a person looses weight through dieting and exercise, the body changes metabolic patterns in order to retain fat. This results in a continuous cycle of dieting and weight gain. This repetitive cycle also significantly contributes to morbidity and mortality as a result of high circulating fat levels in the blood.

This biphasic injection formulation circumvents this vicious cycle. Administration of Lipolyte has the biological property of reducing the number of adipose cells in the treated areas. In combination with exercise and appropriate diet modifications, the ongoing starvation verses feasting cycle is circumvented.

A principal active ingredient used in the present biphasic injection formulation is a phospholipid selected from the group consisting of: lecithin, phosphatidylcholine, 3-sn-phosphatidylcholine, soya (Phospholipon 90), 3-sn-phosphatidylcholine, hydrogenated soya (Phospholipon 90H), (Phospholipon 100H), 3-(3sn)-phosphatidyl)glycerol soya (Phospholipon G), (Phospholipon 90G), dimyristoylphosphatidylglycerol, lysophosphatidylcholine or dipalmitoylphosphatidylglycerol, and physiologically tolerated salts thereof, or a mixture of these compounds. The physiologically tolerated salt of the phospholipid employed is its sodium, potassium or ammonium salt. This active ingredient or active ingredients have the biological properties of causing adipose cells and related tissue to release lipase and related substances. This released lipase, and related substances, have the biological properties of the lysis, destruction, and reduction of the amount of adipose cells in a given region. In addition lipase, and related substances, also has the biological properties resulting in lipolysis of fatty material contained within these adipose cells and adipose tissue.

Another active ingredient used in this biphasic injection formulation is a sclerosing compound. In this invention the sclerosing compound is aqueous sodium chloride. This active ingredient has the biological property of causing a mild tissue inflammatory effect. This inflammatory action has the physiological function of increasing the adherence of subcutaneous layers of skin and fascia resulting in increased skin turgor and stimulation of fibrocyte activity. It is a treatment for disorders of tissue adhesion. Disorders of tissue adhesion often accompany the subcutaneous accumulations of fat which are to be removed. Disorders of tissue adhesion often result from the procedures used for the removal of subcutaneous accumulations of fat. The local derangements of tissue adhesion are often of an unwanted esthetic or pathological nature, and are loose skin, flaccid skin, wrinkles, decreased skin turgor, striae atrophicae, striae albicantes and, a component of cellulite.

Another component of this biphasic injection formulation is a bile salt or a bile acid. These serve as an emulsification component. The physiologically tolerated salt of the bile acid employed is its sodium, potassium or ammonium salt. The bile acid employed is selected from the group consisting of deoxycholic add, cholic add, lithocholic acid, chenodeoxycholic acid, hyodeoxycholic acid, trihydroxycoprostanic acid, ursodeoxycholic acid, taurocholic acid and glycocholic acid, and the physiologically tolerated salts thereof, or a mixture thereof.

Another component of this biphasic injection formulation is an alcohol. The alcohol serves as a clarifier, a solvent, and as a preservative. The alcohol employed is benzyl alcohol. Other suitable solvents are ethanol, propranol, and isopropyl alcohol.

Another component of this biphasic injection formulation is a acidification agent. The acidification agent is hydrochloric acid.

Another component of this biphasic injection formulation is an alkalizing agent. The alkalizing agent is sodium hydroxide.

This biphasic phospholipid based composition of the present invention is useful as a parenteral formulation in treating subcutaneous fat deposits, decreased skin turgor, striae atrophicae, xanthelasma, striae albicantes, and subcutaneous cellulite deposits.

A biphasic phospholipid based composition comprising: a) an effective amount of an active ingredient comprising a phospholipid selected from the group consisting of: 3-sn-phosphatidylcholine, soya (Phospholipon 90), 3-sn-phosphatidylcholine, hydrogenated soya (Phospholipon 90H), (Phospholipon 100H), 3-(3sn)-phosphatidyl)glycerol soya (Phospholipon G), (Phospholipon 90G), dimyristoylphosphatidylglycerol, lysophosphatidylcholine or dipalmitoylphosphatidylglycerol, and physiologically tolerated salts thereof, or a mixture of these compounds. Wherein the physiologically tolerated salt of the phospholipid employed is its sodium, potassium or ammonium salt. This active ingredient or active ingredients having the biological properties of causing the destruction of adipose cells and the lipolysis of the fatty material contained within the treated adipose tissue; b) a bile acid or a bile salt wherein the bile acid employed is selected from the group consisting of deoxycholic add, cholic acid, lithocholic acid, chenodeoxycholic acid, hyodeoxycholic acid, trihydroxycoprostanic acid, ursodeoxycholic acid, taurocholic acid and glycocholic acid, and the physiologically tolerated salts thereof, or a mixture thereof. Wherein the physiologically tolerated salt of the bile acid employed is its sodium, potassium or ammonium salt. That this bile acid or bile salt having the properties of being an emulsifier; c) a sclerosing component; d) water; e) a clarifying component; f) an alkalizing agent; g) and a acidification agent.

A biphasic phospholipid based composition comprising:

a) a lipidic phase comprising:
(i) lecithin or hydrogenated lecithin wherein the concentration is from 0.05% by weight to 50% by weight in the preparation;
(ii) phosphatidylcholine or hydrogenated phosphatidylcholine wherein the concentration is from 0.05% by weight to 50% by weight in the preparation;
(iii) optional lipid soluble components as described below in (e) wherein the concentration is from 0.00% by weight to 50% by weight in the preparation; and
   b) this lipid phase of (a) comprising 0.0 to 100 percent by weight in the preparation;
   c) an aqueous phase comprising:
(i) water wherein the concentration is from 0.0000% by weight to 100% by weight in the preparation;
(ii) sodium chloride wherein the sodium chloride is from 0.00001% by weight to 10% by weight in the preparation;
(iii) benzyl alcohol wherein the alcohol is from 0.00001% by weight to 20% by weight in the preparation;
(iv) hydrochloric acid wherein the hydrochloric add is from 0.00001% by weight to 10% by weight in the preparation;
(v) a bile acid or a bile salt wherein the bile acid, bile salt, or mixture of these compounds is from 0.05% by weight to 50% by weight in the preparation;
(vi) sodium hydroxide wherein the sodium hydroxide is from 0.00001% by weight to 10% by weight in the preparation;
(vii) optional aqueous soluble components described below in (f) wherein the concentration is from 0.00% by weight to 50% by weight in the preparation; and
   d) this aqueous phase of (c) comprising 0.0 to 100 percent by weight in the preparation;
   e) lipid soluble biologically active substances as added to individualized treatment regimens as further selected from the group consisting of nutrients, micro nutrients, vitamins, and drugs; and
   f) aqueous soluble biologically active substances as added to individualized treatment regimens as further selected from the group consisting of nutrients, micro nutrients, vitamins, and drugs.

Lecithin can be used as natural lecithin in purified sterile form or as the more stable hydrogenated lecithin, whereby the formulation is more stable. Lecithin in this invention consists of at least 90 percent of a phospholipid. Lecithin is a phospholipid which serves as a principal factor involved in the transport, regulation, and metabolism of fatty substances. It is a fatty food substance. It is a structural component of every cell in the body. It is an important component of cell membranes. Presently, the phospholipid is administered orally or parenterally as either an intravenous (IV) or subcutaneous (SC) injection.

Presently, lecithin is injected SC for the reduction of subcutaneous fat deposits. This procedure was discovered by Brazilian dermatologist Patricia Rittes and is most commonly called Lipo-Disolve. In April 1999 the FDA approved a Baxter Healthcare Corporation product, Cernevit-12, which contains lecithin for injection. This product is a vitamin delivery system for parenteral nutrition.

A biphasic formulation comprises an aqueous phase and a lipidic phase. This provides for the solubility of both lipid and aqueous soluble components. This formulation then can be a vehicle for the transport of both lipid and water soluble substances to the targeted treatment area.

The properties of such a biphasic formulation will provide conditions which favor the formation of liposomes. Liposomes are small vesicles comprising amphipathic lipids arranged in spherical bilayers. Liposomes may contain many concentric lipid bilayers separated by aqueous channels (multilamellar vesicles or MLVs), or alternatively, they may contain a single membrane bilayer (unilamellar vesicles), which may be small unilamellar vesicles (SUVs) or large unilamellar vesicles (LUVs). The lipid bilayer is composed of two lipid monolayers having a hydrophobic “tail” region and a hydrophilic “head” region. In the membrane bilayer, the hydrophobic “tails” of the lipid monolayers orient towards the center of the bilayer, whereas the hydrophilic “heads” orient towards the aqueous phase.

Liposomes may be used to encapsulate a variety of materials by trapping hydrophilic compounds in the aqueous interior or between bilayers, or by trapping hydrophobic compounds within the bilayer. As such, they are particularly useful to deliver biologically active materials by encapsulating compounds which exhibit poor aqueous solubility or which exhibit unacceptable toxicity at therapeutic dosages. In addition, liposomes may be used to deliver biologically active materials which are at the same time components of the liposome itself.

This biphasic phospholipid based parental composition is appropriate to be delivered by subcutaneous, intravenous and intramuscular injection. A lecithin containing formulation was FDA approved for subcutaneous and intravenous injection in April of 1999 to Baxter Healthcare Corporation.

The compounds of the lipid phase constitute a stabilizer. Additionally, the components of both the lipid and aqueous phase together comprise a mild buffer. The set pH and capacity of this buffer can be adjusted to predetermined amounts by changing the relative amounts of the dosage formulation. This involves the adjustment of the relative amounts of the lipid and aqueous phases. It also involves adjustment of the components of the alkalizing agent, the acidification agent, and the bile acids or their salts. It also involves the adjusting the relative amounts of the components of both the lipid and aqueous phases. This provides for the adjustment of the parameters of this buffering action to be tailored to most suit the given target area undergoing treatment by injection of the formulation. The pH of the preparation is in the range from 6.5 to 9.0, preferably from 6.5 to 8.0, in particular from 6.5 to 7.8.

This invention also comprises a program to facilitate the efficacy of the formulation. The dosage and given formulation injected in a given patient is to be guided by a physician skilled in the art on a case by case basis. Patients may receive multiple injections, appropriately spaced, to a given target area per day. This schedule of injections may be then repeated as per the physicians clinical judgment.

This invention also comprises a method of sub cutaneous injection to minimize patient discomfort and to maintain a sterile procedure. To these ends the area to be treated is first cleaned with an alcohol gel. Then a gel containing a surfactant and the topical anesthetic preparation is applied. The anesthetics include Benzocaine, Lidocaine, and Tetracaine. Included is lecithin as a transdermal transport agent. This is followed by a second layer of the anesthetic preparation. The area is covered with a layer of clear plastic wrap which was known by the trade name Saran Wrap. A heating pad is placed over this area with low heat in order to warm the tissue and facilitate the penetration of the above applied preparations. This heating pad is left on for about 30 minutes. The heating pad and clear plastic wrap are removed. The area is then disinfected with povidone-iodine gel. Multiple injections of Lipolyte preparation is then injected subcutaneously. The treated area is wiped to remove the remaining gel present. A massage cream containing the homeopathic preparation Arnica is applied. Mechanical massage treatment is then applied. The patient is then discharged.

With normal skin turgor, it takes about 35 PSI (pounds per square inch) for a needle to penetrate the skin. With application of the surfactant and the subsequent heat significantly decreases this pressure required for a needle to penetrate the skin. It also increases the efficacy of the topical anesthetic. This significantly decreases the pain upon needle injection. This is important with lipolyte for often more than 100 individual injections are made during a given session. Pain control and disinfection are of significant importance with this procedure.

It has been found that the application of a compression garment to the treated area facilitates both the removal of subcutaneous fat and also the sclerosing action of the formulation. A moderate exercise program is also encouraged and guided by a physician skilled in the art on a case by case basis. Diet modification, education, and counseling is also guided by a physician skilled in the art on a case by case basis. To this end, a diet approximately equal in protein with complex carbohydrates and low fat is encouraged. The combination of injections, compression garment, diet, and moderate exercise has proven to be the most efficacious in the treatment of subcutaneous fat deposits.

In the early clinical application of this invention, and in our original patent application, an anti-inflammatory and anti oxidant component was considered. One such compound was tocopherol. It was postulated that it's effects on healing would limit the possibility of side effects and increase treatment efficacy. To this end, an IV formulation of mixed vitamins, including tocopherol was placed in the preparation. Clinical evaluation revealed that the presence of the vitamins and antioxidants decreased the efficacy of the formulation. Through our research and clinical experience, it was discovered that these vitamins decreased the sclerosing activity of the preparation. The resultant efficacy of the preparation was reduced. Vitamins and tocopherol were removed with resulting increased efficacy and a decrease in such side effects as hematoma formation. This indicated that the positive effects of the mild sclerosing action of the preparation was of greater significance compared with any benefits of antioxidant components. It is clear that for increased efficacy, anti-inflammatory components must not be part of this preparation when used in accordance with this invention.

In the early clinical application of this invention, and in our original patent application, the inclusion of an anesthetic was included in the formulation. One such compound was lidocaine. Initially, a topical anesthetic was applied, with an anesthetic in the preparation to increase the efficacy of anesthesia. When the preparation was used in small amounts, their was not much of a problem. One of the advantages of our invention is the fact that the inflammatory reaction limits the spread of the preparations actions to a localized area around the injection site. As a result of this localization, it became possible to use relatively high doses of the product within a given session. Total dosages of over 100 ml became quite common. Also, the rest period between treatment administrations was able to be reduced from a minimum of 10 days, as described in previous literature, to just a day or two. The result was the systemic release of higher and higher doses of local anesthetics. Some of the patients started becoming sick. Symptoms included nausea, vomiting, diarrhea with dizziness.

This problem was remedied by complete removal of anesthetics from the injection preparation. A compounding pharmacy came up with an advanced topical anesthetic combination of several anesthetic drugs with a transdermal transport system. This preparation is not part of this invention. But it's use has allowed the total removal of anesthetics from the preparation. It has been found to provide adequate anesthesia without anesthetics included in this inventions formulation. Anesthetics are no longer a component of this inventions injection formulation.

As stated above, the lecithin based compositions marketed have some efficacy in the treatment of subcutaneous fat deposits. It has been found that the biphasic dosage formulation based on lecithin and aqueous sodium chloride described above has enhanced efficacy in the treatment of subcutaneous fat deposits. Additionally, this formulation has shown efficacy in the treatment of other disorders as disclosed above.

The efficacy of treatment of subcutaneous fat deposits can be further enhanced by the addition of including such injections into a program including diet, exercise, and the wearing of a compression garment as guided by a physician skilled in the art on a case by case basis.

### OPERATION OF INVENTION AND IT'S ALTERNATIVE EMBODIMENTS

The present invention relates to a biphasic nutrient and micro nutrient composition which includes phospholipids and aqueous sodium chloride. The biphasic nature of this composition promotes the formation of liposomes in solution. The composition is useful for treatment of subcutaneous fat deposits, striae albicantes, striae atrophicae, cellulite, and decreased skin turgor, and the different etiologies therewith.

The main active ingredients are phospholipids and aqueous sodium chloride. Phospholipids and their pharmaceutically acceptable derivatives and related compounds as listed elsewhere in this application, have the biological properties of stimulating lipase production in adipose tissue. Lipase and it's related compounds stimulate the lysis, destruction, and reduction of the amount of adipose cells. Lipase also stimulates the lipolysis of fatty material contained within adipose tissue.

In the 1990's the World health Organization (WHO) identified obesity as an epidemic of mass proportions. The United States Center for Disease Control (CDC) estimates that about 300,000 Americans die annually from obesity-related illnesses. The United States Surgeon General has determined, in 2004, that there is now an epidemic of adult onset diabetes in children with an onset of about age 10. It is feared that we may be approaching a time where the present young generation may start passing away before their parents in significant numbers. Obesity is a serious and intractable health hazard. The vitality of our nation is at stake.

The underlying problem is that the body has an internal set point of how much fat composition is optimal. This set point is basically determined by the numbers of adipose cells present. This was determined by both genetic and environmental factors. The environmental factors include the behavior of a person's mother during pregnancy, behavior patterns during growth spurts during early childhood, and at puberty, and continuing behavioral patterns into adulthood.

The problem is further compounded in that obesity causes obesity. Changes in the production and or clearance of certain hormones is associated with increasing body mass and regional fat distribution. These hormonal changes promote further weight gain and affect the distribution of fat in humans. Included are high blood levels of insulin and cortisol. It also includes low blood levels of growth hormone. Testosterone blood levels are also altered, with them being elevated in women and depressed in men. These metabolic abnormalities promote excess fat deposits and a tendency to cause these deposits in body areas where it is harder to loose such fat deposits. These patterns are well known and are different in men and women.

There is even a very viscous hormonal feedback cycle involving cortisol. In the field of Psychoneuroendocrinology, it has been known that high cortisol levels most likely is involved in the genesis and character of primary mood disorders. There is also known to be a relationship between chronic stress and depressive disorders. Additional evidence suggests that prolonged high levels of cortisol can result in structural neuropathology resulting in more lasting behavioral change. See Kaplan and Sadock et al., Comprehensive Textbook of Psychiatry ed. V, volume 1 pages 105-106 (1989). And this cycle viscously accelerates as obesity releases more cortisol and this worsens underlying psychiatric pathology resulting in obesity. A very dangerous, unhealthy, and insidious pathological trap with significant crossover into non psychiatric pathological states and diseases.

When a person looses weight through dieting and exercise, the body changes metabolic patterns in order to retain fat. This results in a continuous cycle of dieting and weight gain. This repetitive cycle also significantly contributes to morbidity and mortality as a result of high circulating fat levels in the blood.

This biphasic injection formulation circumvents this vicious cycle. Administration of Lipolyte has the biological property of reducing the number of adipose cells in the treated areas. In combination with exercise and appropriate diet modifications, the ongoing starvation verses feasting cycle is circumvented.

The dispersion of the biphasic injection formulation of the present invention is useful in increasing the efficacy of subcutaneous injection treatment of subcutaneous fat deposits. This allows a more evenly and controlled dispersion of the formulation of the components within the adipose tissue. Tendencies of clumpy and uneven distribution of monophasic formulations is eliminated, along with the corresponding uneven reduction of subcutaneous fat. Additionally, this formulation can be used in the treatment of uneven, toxic fat deposits know as cellulite. Additionally, the even distribution of this formulation permits lower amounts of the biologically active substances to be injected for a given response.

The characteristic of this formulation allowing lower amounts of active material present in a given treatment permits in a more controlled reduction of fat deposits. This is of significance in that the reduction of these fat deposits is accomplished by the lysis and destruction of the adipose tissues present. Such destruction of even unwanted cells has further pathologic sequela and the more gradually and evenly such destruction can be accomplished diminishes the associated morbidity. In addition, with slow, gradual reduction of fat deposits, the physiologic and hormone mediated response of the bodies feast verses famine cycle can be circumvented.

The lysis of subcutaneous adipose cells constitutes an injury physiologically. A stress response results which can result in profound metabolic abnormalities. It is for this reason that these physiological injuries of lysis proceed in a highly controlled manner as facilitated by the unique composition and properties of this biphasic injection formulation. As a result a more limited stress load can be imposed at during a given time period, thus circumventing a significant stress response.

A stress response results in profound metabolic abnormalities following the release of inflammatory mediators and the development of an abnormal “stress induced” hormonal environment. An increase in the proinflammatory cytokines TNF, II.sub6, IL.sub.8 and increased oxidant activity, result in further increased cell damage and protein degradation. The cell damage and protein degradation result in an increase in endogenous catabolic hormones. Catalysis of adipose tissue is an objective of treatment with this formulation. However, it is desirable that such catabolic action be localized, controlled, and self limiting.

An increase in the endogenous hormones, such as catechols, cortisol, and lipase, and a decrease in normal endogenous anabolic activity can lead to a large net protein loss, if this occurs in a large quantity. In addition, a profound increase in cell energy demands arises, markedly increasing the need for nutrient utilization, while at the same time energy production becomes very inefficient. This degree of increase in metabolic rate varies with the degree of systemic injury. To this end it is imperative that the quantity of systemic injury be limited and highly controlled. The properties of this biphasic injection formulation greatly facilitate these ends by more even dispersion of the involved active substances. At the same time, the dispersion of this formulation is localized to the near vicinity of the injection site due to the inflammatory response caused by the sclerosing agent. These two actions together allow the catabolic reactions to be more controlled, localized, and self limiting. This allows the removal of adipose tissue with greater control by breaking it's removal into much more controlled, limiting steps. As a result, this treatment can proceed with little or no systemic side effects.

Peak hypermetabolism and increased energy demand begins immediately post injury. It is therefore appropriate to give nutrition support to the treated area involving large amounts of tissue. An entire spectrum of abnormalities can be seen post injury including infection and also inflammation as a manifestation of the host “stress response.” If uncontrolled, this process becomes auto destructive. Support of the metabolic machinery is necessary to prevent further spread of this process.

Lean body mass (LBM) makes up 70% of body weight, with 75% of LBM being water and 20% of the LBM being protein. Almost all protein content of the body is in the LBM compartment. Each protein molecule has a functional role in maintaining homeostasis.

The degree of lean body mass, or body protein loss, in a catabolic state is correlative to morbidity and mortality. LBM loss exceeding 10% of total, can occur within a week after severe injury, despite provision of appropriate macro nutrients, carbohydrates, fat, and protein. A loss of lean mass exceeding 10% of total body protein will result in an immune deficiency state. When losses exceed 15% of body protein, there is also a marked increase in infections, severe weakness, skin breakdown (pressure sores), and the absence of wound healing. A loss of LBM exceeding 40% is usually fatal.

Excess oxidant release is known to produce further tissue injury. Oxidants are very unstable metabolites of oxygen released by inflammatory cells when activated. The oxidants injure tissue by reacting with the cell membrane lipid layers and tissue proteins, thereby producing biochemical damage via the oxidation process. Oxidation of lipids, particularly those of the cell membrane, result in a self-perpetuating process known as lipid peroxidation. Lipid peroxidation results in an alteration in cell membrane function. Post injury red cell hemolysis is caused by oxidant cell membrane injury. However, it is the goal of this biphasic injection formulation to cause peroxidation of the adipose cell membranes, but to limit such damage to them. Further lipolysis of the released contents of the lysed adipose cells in vitro is also a goal of this biphasic injection formulation. However, it is also a goal to limit such peroxidation and lipolysis to adipose cells and their contents only. The properties of the biphasic composition of this invention limit these processes to small localities surrounding the injection sites. This is the result of the controlled dispersion properties of this formulation. In. addition, the formulation contains sodium chloride, a mild sclerosing agent. This sclerosis of localized tissue has the biological effect of compartmentalizing these chemical processes to small confined local areas, thus limiting the spread of these chemical reactions. This prevents the production of a self perpetuating reaction which will then spread to other areas and tissues.

Proteins attacked by oxidants will be denatured, thus rendering them inactive with respect to their normal biological functions. This becomes of particular concern with respect to enzymes and interstitial proteins. However, the sclerosing action of the formulation results in some of the interstitial proteins adhering in what may be described as similar to scar tissue formation. As a result, the catabolic reactions tend to become compartmentalized as described above. Many other processes are also affected by oxidant damage.

The inventors have devised a therapeutic biphasic phospholipid based injection formulation useful for treating patients with subcutaneous fat deposits, striae albicantes, striae atrophicae, cellulite, and decreased skin turgor, and the different etiologies therewith. Each of the components serves to provide nutrients and other biological functions within the physiological system of the patient.

Lecithin is the most abundant phospholipid in the body. It is a fatty food substance. It is a structural material in every cell of the body. It forms 30% of the dry weight of the brain. It is an important constituent of endocrine glands, muscles, the heart, kidneys, liver, and blood. It occurs naturally in many foods including vegetable oils, eggs, whole grain cereals, soybeans, liver, and milk. It is also synthesized in the body, primarily in the liver.

It has properties which allow it to emulsify oils and cholesterol, making them soluble and transportable in aqueous media. These proprieties allow it to break up cholesterol and other lipid compounds into smaller particles more easily transported, assimilated, and metabolized. As such, it is included in the bile produced by the liver which makes fats soluble in the small intestine, and after these fats are absorbed through the intestinal wall, the phospholipid is included in the enterohepatic recirculation recycling process. It is an extremely important factor in the digestion and oxidation of fats. The disease process atherosclerosis is characterized by increased cholesterol and decreased phospholipids in the blood.

Lecithin has been used in the treatment of atherosclerosis, xanthelasma, anxiety, depression, immunodeficiency, acne, eczema, psoriasis, diabetes, exhaustion, and impotence. It is a primary source of phosphatidylcholine. Phosphatidylcholine is from less than 10% to over 96% of lecithin.

Phosphatidylcholine is a primary dietary source of choline, is composed of a phosphate group, 2 fatty acids, and choline. It is the composition of the fatty acids that determines it's value in promoting health. After ingestion, most is broken down into choline, glycerol free fatty acids, and the phosphate group. Some is incorporated intact into cell membranes. However, most cell membrane phospholipids are synthesized from these and other components for use in cell membranes. Although choline can be manufactured in humans form methionine or serine, it has recently been designated an essential nutrient.

Choline is required for the proper metabolism of fats and facilitates the movement of fats in and out of cells. In the human body, it is a methyl donor. This is an extremely important metabolic step in the functions of the liver and other metabolic machinery of the human body. It is vital in liver function due to it's role in the lipotropic effect which involves the export of fat from the liver. Without adequate choline, fats become trapped in the liver and as a result block many metabolic steps. Stagnation of these key metabolic pathways leads to serious liver disorders including cirrhosis. The functioning of similar metabolic pathways is vital for the transport of fats into and out of adipose tissue, and important consideration in the operation of this biphasic injection formulation.

Choline is needed for cell membrane integrity. It plays a critical role in the manufacture of primary cell membrane components including phosphatidylcholine and sphingomyelin. It is a main structural support of cell membranes. Cell membranes are dynamic molecular sheets on which most biochemical life processes occur. Phosphatidylcholine comprises about 40% of the total membrane phospholipids. It is important for homeostatic regulation of membrane fluidity. It is an important mediator of prostaglandin and eicosanoid cellular messenger functions and for support of signal transduction from the cell's exterior to its interior.

The operation of cell membranes is the key to all life processes as we understand them. These functions are crucial to the functioning of this biphasic injection formulation. The chemical signaling from the exterior to the interior of the cell is vital in the triggering of the release of lipase, and it's related materials, upon which a basic function of this biphasic injection formulation depends. But these functions extend far beyond the initial scope of this biphasic injection formulation for the treatment of subcutaneous fat deposits.

The proper functioning of cell membranes is vital for the cell to “speak or communicate” with other cells in it's proximate environment. One of the primary biological failures behind the cluster of diseases known as cancer is a failure of cells to normally communicate within their proximate environment. This communication is necessary to regulate the rate of cell division. Such a failure of communication from cell to cell results in unregulated cell division where each cell has become “an island unto itself.” Though there are many pathological conditions underlying cancer, all cancers share this fundamental communication failure. As a result, choline is vital in the normal function of cells and probably plays significant roles in the pathology of cancer. There is a strong possibility that proper maintenance of normal choline composition of cell membranes will play a role in the prevention and treatment of many cancers.

Choline is essential in the synthesis of acetylcholine which is essential in many brain, neuronal, and other chemical processes of life.

Phosphatidylcholine is the main lipid constituent of the lipoprotein particles circulating in the blood. It increases the solubility of cholesterol thus lowering cholesterol levels, removing cholesterol from tissue deposits, and inhibits platelet aggregation. Alteration of all of these processes contribute to atherosclerosis.

Phosphatidylcholine's amphipathic properties make it a necessary micellizing constituent of bile. It has surfactant properties making it a protector of the epithelial-luminal interfaces of both the lungs and GI tract. It is a precursor for other phospholipids and their components as described above.

The health of the cell membrane is synonymous with health of the entire organism. Toxins have an affinity for fatty acids; they literally take up residence in the lipid environment and in so doing, weaken and disrupt metabolic processes. The probable result is early apoptosis, premature death of the cell. Generally, normal mitosis provides for new cellular growth to maintain the health of the body. However, toxicity's affinity for lipids can easily redistribute toxins and diseased toxic lipids into new growth. In a healthy state with adequate glutathione and ascorbate to bind toxins before they take up new residence, the body can keep up with the bad guys under control. However, if defenses are weak, toxins can continually be redistributed and eventually hide in the CNS and bone where regeneration is slow.

Detoxification of neurotoxins requires that the cell membrane is nourished with balanced essential fatty acids and supportive phospholipids. Phosphatidylcholine is the main lipid constituent of cell membranes and assists the 33,000 square meters of liver cell membrane to be protected from toxicity and infection. The liver should play a pivotal role in detoxification but due to its fatty acid content and the lipid soluble characteristics of neurotoxins, lipid based interventions, such as possible with this biphasic injection formulation, are required to impact toxic burdens. Once the liver has been damaged it can no longer metabolize fats normally. Pools of lipids are then deposited within the hepatocytes throughout the liver. Beta oxidation of fatty acids is suppressed impairing detoxification and prostaglandin production. However, research has shown that phosphatidylcholine protects the liver against damage from alcohol, pharmaceuticals, environmental pollutants, xenobiotics, and infection due to viral, bacterial, and fungal manifestations.

The widespread biological properties of lecithin and phosphatidalcholine indicate the importance of these micro nutrient compositions of this biphasic injection formulation to the basic life processes and maintenance of homeostasis. Additionally, these properties are also vital to protect the body from possible systemic complications from the injury of lysis of adipose issue. It additionally mitigates against the further spread of this damage from the localized treatment area. It also facilitates the restoration of the remaining cells to a normally healthy state. Additionally, toxins developed are relatively contained within the small treated area.

Hydrogenated lecithin and phosphatidalcholine are simply more stable variants of their related compounds.

Lysolecithin is a lecithin molecule form which the alpha fatty acid has been removed. It has strong hemolytic properties and exists in trace amounts in the pancreas. A lysophosphatide, as in lysophosphatidylcholine, also has one fatty acid molecule removed. It would as a result also be hemolytic. Both of these compounds are most likely involved in the lipolysis of the adipose cells.

Lysophosphatides are produced by the action of injected cobra venom on phospholipids. Their resulting hemolytic properties are part of the pathological response to cobra envenomation. This hemolysis action is the result of disruption of the cell membrane of the red blood cells. This action closely approximates the actions involved in lipolysis. It is an action of the properties of the lysophosphatides to include lipolysis of adipose cells. In addition to the stimulation and release of lipase products, these lysophosphatides directly contribute to this action.

It was during the academic research done for our first patent application that we found the relationship between lecithin, cobra venom, and lysolecithin. As described above, these actions are antagonistic. When the body absorbs lecithin, it is broken down into glycerol and fatty acids. It is then reassembled into pure lecithin on the opposite side of the bowel lumen. The body “protects itself” from the intrusion of lysolecithin. Lysolecithin is toxic. It is the actual chemical damaging the body by cobra envenomation. The cobra venom itself is like a proto drug. It is not directly active on the human tissue. It clips off the alpha fatty acid from lecithin, forming lysolecithin. The lysolecithin then causes the hemolysis.

Understanding of this relationship is important for the optimal design of such a medication as in this invention. Most inventors previously have focused on the lecithin component. But lysolecithin is a contaminant in commercial lecithin. It was never intended to be present within the body. We believe it is probably the most active component involved in lipolysis. We are here balancing two antagonistic effects. The lipolysis of fat cells and the hemolysis of red blood cells. In order to optimize this effect, the active substances must be in most contact with fat cell membranes and in least contact with red blood cell membranes. This is the exact effect caused by the inflammatory reactions induced by the sclerosing compound sodium chloride. Addition of anti inflammatory agents will reduce, if not stop, the inflammatory reaction. This will decrease the separation of these two antagonistic effects. It is a key component to the increased efficacy of our formulation over those previously developed.

It is important that the reader understand that these actions are diametrically opposed to the protective, homeostatic, anti toxic effect of the phospholipids. This biphasic injection formulation functions by the action of both of these antagonistic qualities. Additionally, the sclerosing actions of other components “wall off” these sites of reaction to make them self limiting and confined to a controlled area. The management of these antagonistic actions of such a preparation as embodied by this biphasic injection formulation takes the skill and clinical experience of a physician with experience in such art.

Sodium chloride is a salt that is ubiquitous to life. Sodium is one of the most commonly transferred ion through the gates of various membranes of cells. It is the most common ion used in all of the “switches” of the body. Most neurotransmitters upon binding to their respective synaptic sites involve the shifting of sodium across a membrane. It is also a mild sclerosing agent and is used as such in this biphasic injection formulation. It is also a component of the buffering action of this biphasic injection formulation.

The components of the buffering action of this biphasic injection formulation include sodium hydroxide, hydrochloric acid, water, phosphatidylcholine, hydrogenated phosphatidylcholine, lecithin, lysophosphatidylcholine, hydrogenated lecithin and the bile salt or acid. These components also comprise a stabilizer.

As stated above, the subcutaneous fat removal injection products currently marketed are primarily monophasic lecithin based mixtures of lipids. The biphasic nature of this biphasic injection formulation allows better and more even distribution of biologically active substances. The sclerosing action limits the actions of the biphasic injection formulation to highly predetermined and controlled locations. The actions are thus localized and self limiting. Components of this biphasic injection formulation promote the healing of remaining tissue and reduce the production and dissemination of toxic metabolites. Further, the above mentioned sclerosing action promotes increased skin tone and turgor, therefore decreasing skin sagging and wrinkling resulting from the use of currently marketed preparations. In addition, the components of the lipid and aqueous phases, in and of themselves have therapeutic properties valuable in the treatment of many human disease conditions as elaborated above.

The preparations of the invention are described in Hexsel et al, Boderke et al, and in Investigations on Mixed Micelle and Liposome Preparations for Parenteral Use Based on soya Phosphatidylcholine in Eur. J. Biopharm 40(3) (1994) by Manfred Durr, Jorg Hager, and Joseph Peter Lohr, incorporated herein by reference. Processes for producing the preparations are also described in European patent applications EP 0 470 437 or EP 0 615 746, incorporated herein by reference.

The preparations of the invention are produced, for example, by mixing sterile water with the alcohol and the sodium hydroxide. The amounts are as indicated above. The bile salt or acid is then added and the mixture is mixed until clear. The phospholipid is then added and the mixture mixed until clear. If needed, more alcohol may be added until the mixture is optically clear. Hydrochloric acid is added drop wise until the pH is 7.6. The remaining water is added in the form of sterile 0.45 normal sodium chloride solution.

Production of the preparations of the invention can after addition of the water be promoted by extrusion, high-pressure homogenization and/or ultrasound treatment.

The preparations may also comprise colloidal structures such as micelles or mixed micelles. These structures have a particle diameter of from 1 to 50 nm. They consist of bile acid and phospholipid. The mass ratio of bile acid to phospholipid is in % by weight from 0.1:2 to 2:1, preferably from 1:2. The phospholipid concentration in the colloidal structures in the medicaments is from 5% by weight to 50% by weight. The colloidal structures are produced for example by dissolving the bile acid in aqueous sodium chloride as normal saline, making the solution somewhat alkaline. The phospholipid is then dispersed therein. Filtration may be carried out.

Administration is a follows. The skin of the treated area is cleansed with an alcohol gel. A surfactant is rubbed into the skin. A topical anesthetic is applied. This gel is made by a compounding pharmacy and contains active ingredients: Benzocaine, Tetracaine, Lidocaine, and lecithin. The area is covered with clear plastic wrap and a heating pad is applied over this plastic. The site is warmed approximately 30 minutes to develop maximum anesthesia and softening of the skin. Anesthesia is tested by pinprick. Additional anesthetic is applied as indicated. The area is then disinfected using a povidone-iodine wash or gel. Lipolyte is drawn into 5 cc syringes and a fine needle is applied. The preparation is injected from just subcutaneous to up to about 1 cm. About 0.5 cc is injected per site. Up to about 10 individual injections are administered with one syringe. As needed, more syringes of lipolyte are used. It is common to inject over 100 cc in a given session. The skin is then cleaned. A massage cream containing the homeopathic preparation Arnica is applied. Mechanical massage treatment is then applied. The patient is instructed to wear a compression garment. The patient is discharged and followed up by the attending physician. In event of hematoma or other side effects, these are managed with conservative care.

While I believe the operations of this invention occur as described above, I don't wish to be limited and or bound by these explanations.

### PREFERRED EMBODIMENTS

The first preferred composition are those of the following general formula:
Sterile water 500 ml;
Benzyl alcohol 20 ml;
Sodium Hydroxide 1.25 grams;
Deoxycholic acid sodium salt 23.75 grams;
Phospholipon 90 G 48 grams;
Hydrochloric acid titrated to a solution pH of 7.6. This constitutes about 35 drops, with 15-16 drops per ml. This totals about 2 to 2.5 ml.
0.45 normal sterile aqueous sodium chloride solution 500 ml.
The phosphatidylcholine used is Phosphplipon 90G by American Lecithin Company or it's commercial equivalent. It approximately comprises about 94% or more of the lipid phase. The remaining 0.0-6% of the lipid phase is comprised of lysophosphatidylcholine;
Hydrogenated variants of the above compounds comprise 0.0-100% of the given compound.

This preferred composition is suitable for primarily subcutaneous injection or infusion only. It can also be applied transdermal. It contains hemolytic and lipolytic components that must not be delivered intravenously or intramuscularly except under rare conditions as deemed necessary by a physician. This composition of the formulations of this biphasic injection formulation is to be used primarily for treatment of subcutaneous fat deposits, cellulite deposits, decreased skin turgor, striae atrophicae, and striae albicantes.

For intra-articular, intraperitoneal, intramuscular injection, or for short IV infusions, the formula must be modified. A much purer form of phosphatidylcholine such as Phospholipon 100H by American Lecithin Company or it's commercial equivalent must be substituted.

Boderke describes the application of Essentiale by the tumenescence technique in line 71 of their patent. Lipolyte can be used in the same manner, but with some modifications. I use Borderke's wording as appropriately modified. On application to large areas, administration of Lipolyte by means of the tumenescence technique is to be regarded as a particularly suitable method. This entails in the first step up to 8 liters of a saline solution being infiltrated into the adipose tissue, and the adipose tissue being mobilized. Their will be a small amount of anesthetics used in the solution. It must be limited in amount in order to avoid excessive systemic absorption resulting in toxic side effects such as we have experienced in the past with large infusion amounts on injection. Substances with anti-inflammatory activity must not be included, as this will counter act the advantage of the inflammatory reaction in treating the obvious disorders of tissue adhesion caused by such a procedure. By allowing the inflammatory reaction, there will be much improvement in tissue adhesion compared with a mixture suppressing the inflammatory reaction. The result will be much less tissue sag and looseness which often accompany liposuction procedures. The main mass of the fat is then sucked out. Addition of Lipolyte to the infiltrate assists liposuction by medicinal lipolysis. The infiltration method allows particularly good exposure of Lipolyte in the target tissue.

Percutaneous administration is also claimed, in various carrier media and with use of various aids, for example iontophoresis.

Simultaneous introduction of the preparations and pharmaceutical forms employed according to the invention can also take place in particular applications via a tumescence method which makes use of the hydrostatic pressure in order to ensure uniform distribution.

Percutaneous administration is also possible, which can take place in various carrier media such as creams, ointments, gels, hydrogels, lotions or pastes, and with use of various aids, for example, iontophoresis or phonophoresis.

Suitable preparations and pharmaceutical forms are, for example, suspensions, emulsions or injectable solutions, and products with protracted release of active ingredient, in the production of which conventional aids such as are used. The preparations can also be in the form of a concentrate, dry substance or lyophilizates, in order to increase the stability for example.

It is important that the reader understand that the phosphatide compounds taken orally in foods and supplements most likely contain some lysophosphatide breakdown contaminants. However, during the transmural transport of these phosphatides across the small intestine lumen, they are broken down into choline, glycerol free fatty acids, and the phosphate group. These components are then reassembled into the phosphatide compounds needed without forming lysophosphatides in the process.

In our academic research on the initial patent application, we discovered that lysolecithin is hemolytic. It is also not naturally brought into the body. It is considered a contaminant within normal lecithin. The more pure forms of lecithin have lower amounts of lysolecithin. It's hemolytic properties probably constitute a significant, if not, the majority of the lipolysis action attributed to lecithin.

The combined actions of lipolysis and sclerosis of our invention opens a new approach to the use of these types of medications. By limiting the exposed tissue by the inflammatory reaction, it may be possible to use lecithin preparations containing more amounts of lysolecithin without causing side effects such as hematomas. This is an avenue for future research from which this invention may lead. In order to do this, the sclerosing action may have to be increased, Our invention only uses a mild amount of tissue sclerosis. However, increasing sclerosis may permit future formulations which have much more efficacy in removal of fat deposits. But this must be the study of future clinical research. However, this invention shows that such improvements are likely to be possible.

The subcutaneous injection of lysophosphatide variants circumvents this protection mechanism of the body, allowing the deposit of lipolytic compounds for therapeutic purposes. But the introduction of these normally dangerous compounds is done in a highly controlled and self limiting manner as described above.

Many variations of this preferred embodiment are possible. These other embodiments of the biphasic injection formulation will be apparent to those skilled in the art from consideration of the specification and practice of the invention disclosed herein. It is intended that the specification be considered exemplary only, with the true scope and spirit of the invention being indicated by the attached claims.

### CONCLUSION, RAMIFICATIONS, AND SCOPE

Accordingly, the reader will see that the biphasic injection dosage formulation of this invention can be used to treat subcutaneous fat deposits, can be used to increase skin turgor, and can be used to treat subcutaneous cellulite. Furthermore, this invention comprises a coordinated program of treatment which comprised the injections, wearing a compression garment, diet modification, and an exercise program. These features increase the efficacy of treatment over just injections alone. Furthermore, the biphasic injection dosage formulation has the additional advantages in that:
it permits the thinning of the phospholipid mixture prior to injection;
it provides for increased and more uniform dispersion of the phospholipid in the target tissue;
as a result of more uniform dispersion of the injected dosage formulation, there is a marked reduction in the uneven dissolution of subcutaneous fat deposits as compared with previous products. As a result, the tendency to leave uneven, lumpy deposits under the skin is eliminated;

it provides a means of treatment of cellulite in a human in need of such treatment;
it provides a means of treatment of striae atrophicae in a human in need of such treatment;
it permits the predetermining of the pH of the preparation;
it provides a means of treating human ailments responsive to the biological properties of it's lipophilic components;
it provides a means of treating human ailments responsive to the biological properties of it's aqueous components.

Although the description above contains many specificity's, these should not be construed as limiting the scope of the invention but as merely providing illustrations of some of the presently preferred embodiments of this biphasic injection formulation. For example, the relative amounts of the lipid and aqueous compounds may change; biologically active materials may be carried by the formulation, etc. Other embodiments of the biphasic injection formulation will be apparent to those skilled in the art from consideration of the specification and practice of the invention disclosed herein. It is intended that the specification be considered as exemplary only, with the true scope and spirit of the invention being indicated by the appended claims and their legal equivalents, rather than by the examples given.

We claim:

1. A method for removing subcutaneous accumulations of fat, for the sclerosis of involved tissue, and comprising the administration of an efficacious amount of a preparation comprising a) at least one phospholipid, b) at least one bile acid or bile salt, c) a sclerosing component and d) water.

2. The method of claim 1, wherein the preparation comprises a biphasic dosage formulation comprising a) an aqueous phase and b) a lipidic phase.

3. The preparation of claim 2, wherein the aqueous phase comprises an aqueous solution of a) water b) hydrochloric acid c) sodium hydroxide d) at least one alcohol, e) at least one bile acid or one bile salt, f) a sclerosing component.

4. The aqueous solution of claim 3, wherein the alcohol is benzyl alcohol.

5. The aqueous solution of claim 3, wherein the alcohol is a clarifying component.

6. The preparation of claim 2, wherein the lipidic phase comprises a phospholipid.

7. A method for the treatment of adipose tissue disorders which are local derangements of fat distribution, the method comprising the removing of subcutaneous accumulations of fat in accordance with the method of claim 1.

8. A method for the regression of adipose tissue tumors comprising the removing of subcutaneous accumulations of fat in accordance with the method of claim 1.

9. The method of claim 7, wherein the local derangements of fat distribution are of an unwanted esthetic or pathological nature, and are lipedemas, lipomatosis of the abdominal walls, dermatopanniculosis deformans, xanthelasma, piezogenic modules, adipose tissue disorders, Dercum's disease, Madelung's neck, striae albicans, or cellulite.

10. The method of claim 1, wherein the phospholipid employed is one of the following compounds: 3-sn-phosphatidylcholine, soya (Phospholipon 90), 3-sn-phosphatidylcholine, hydrogenated soya (Phospholipon 90H), (Phospholipon 100H), 3-(3sn)-phosphatidyl)glycerol soya (Phospholipon G), dimyristoylphosphatidylglycerol, lysophosphatidylcholine or dipalmitoylphosphatidylglycerol, and physiologically tolerated salts thereof, or a mixture of these compounds.

11. The method of claim 10, wherein the physiologically tolerated salt of the phospholipid employed is its sodium, potassium or ammonium salt.

12. The method of claim 1, wherein the sclerosing component is sodium chloride.

13. The method of claim 1, wherein the bile acid or bile salt is an emulsification component.

14. The method of claim 1, wherein the bile acid employed is selected from the group consisting of deoxycholic acid, cholic acid, lithocholic acid, chenodeoxycholic acid, hyodeoxycholic acid, trihydroxycoprostanic acid, ursodeoxycholic acid, taurocholic acid and glycocholic acid, and the physiologically tolerated salts thereof, or a mixture thereof.

15. The method of claim 14, wherein the physiologically tolerated salt of the bile acid employed is its sodium, potassium or ammonium salt.

16. The method in claim 1, wherein the bile add, bile salt, or mixture of these compounds is from 0.05% by weight to 50% by weight in the preparation.

17. The method of claim 1, wherein the phospholipid concentration is from 0.05% by weight to 50% by weight in the preparation.

18. The method of claim 1, wherein the sclerosing component is from 0.00001% by weight to 10% by weight in the preparation.

19. The preparation of claim 3, wherein the alcohol is from 0.00001% by weight to 20% by weight in the preparation.

20. The preparation of claim 3, wherein the hydrochloric acid is from 0.0001% by weight to 10% by weight in the preparation.

21. The preparation of claim 3, wherein the sodium hydroxide is from 0.0001% by weight to 10% by weight in the preparation.

22. The preparation of claim 3, wherein the water is from 0.0001% by weight to 100% by weight in the preparation.

23. The method of claim 1, wherein the phospholipid is present in an effective amount and having the biological properties of causing adipose cells and related tissue to release lipase and related substances, said lipase and related substances having the biological properties of the lysis, destruction and reduction of the amount of adipose cells in a given region, said lipase and related substances having the biological properties resulting in lipolysis of fatty material contained within said adipose cells, resulting in reduction of the number of adipose cells present in a treated area, and resulting in the reduction of the amount of adipose tissue present in a treated area.

24. The method of claim 1, wherein the sclerosing component is present in an effective amount and having the biological properties of causing an inflammatory reaction within the tissue exposed to this component.

25. The inflammatory reaction within exposed tissue of claim 24, wherein this inflammatory reaction is a method for the treatment of loose or flaccid tissue disorders which are local derangements of tissue adhesion which often accompany the subcutaneous accumulations of fat which are to be removed.

26. The inflammatory reaction within exposed tissue of claim 24, wherein this inflammatory reaction is a method for the treatment of loose or flaccid tissue disorders which are local derangements of tissue adhesion which often result from removal of subcutaneous accumulations of fat.

27. The method of claim 25, wherein the local derangements of tissue adhesion are of an unwanted esthetic or pathological nature, and are loose skin, flaccid skin, wrinkles, decreased skin turgor, striae albicantes and, a component of cellulite.

28. The method of claim 1, wherein the preparation is administered by subcutaneous, intra-articular, intraperitoneal, intramuscular injection, short infusions, infusion, or by use of the tumenescence technique.

29. The hydrochloric acid of claim 3, wherein the hydrochloric acid is an acidifying agent.

30. The acidifying agent of claim 29, wherein the pH of the preparation is set by predetermining the relative concentrations of said acidification agent.

31. The sodium hydroxide of claim 3, wherein the sodium hydroxide is an alkalizing agent.

32. The alkalizing agent of claim 31, wherein the pH of the preparation is set by predetermining the relative concentrations of said alkalizing agent.

33. A method of treating subcutaneous adipose tissue accumulation in a humans in need of such treatment comprising: a) injection of the preparation of claim 1, b) application of a compression garment, c) a predetermined exercise program, and d) a predetermined diet regimen.

34. The method of claim 33, wherein said compression garment is to be worn from 0 to 24 hours in a 24 hour time period.

35. The method of claim 33, wherein said compression garment is to be worn for a predetermined number of 24 hour time periods.

36. The method of claim 33, wherein said exercise program is to be predetermined by a practitioner skilled in such art.

37. The means of claim 33, wherein said diet regimen is to be predetermined by a practitioner skilled in such art.

38. Lysophosphatidylcholine is a significant contributor to the lipolysis action of this preparation.

39. A method of injection as in claim 33 wherein the method of injection comprises the following steps: a) cleansing of the treated area by an alcohol gel; b) application of a surfactant; c) application of a topical anesthetic; d) application of localized therapeutic heat; e) disinfection; f) sub cutaneous injection; g) application of therapeutic mechanical massage; and h) application of a topical healing cream.
